(19) United States
(12) Patent Application Publication
(10) Pub. No.: US 2024/0043753 A1
(43) Pub. Date: Feb. 8, 2024

(54) METHOD FOR PRODUCING GASOLINE ALTERNATIVE FUEL AND GASOLINE ALTERNATIVE FUEL

(71) Applicant: HONDA MOTOR CO., LTD., Tokyo (JP)

(72) Inventor: Takuma ARAI, Saitama (JP)

(21) Appl. No.: 18/349,182

(22) Filed: Jul. 10, 2023

(30) Foreign Application Priority Data
Aug. 8, 2022 (JP) 2022-126383

(51) Int. Cl. C10G 2/00; C07C 1/24; C07C 2/06; C07C 4/02; C10L 1/06

(52) U.S. Cl. CPC C10G 2/50; C07C 1/24; C07C 2/06; C07C 4/02; C10L 1/06; C10G 2400/02; C10G 2300/1022; C10L 2290/24; C10L 2290/543

(57) ABSTRACT

Provided is a method for producing a gasoline alternative fuel, comprising synthesizing a crude oil by a Fischer-Tropsch method, distilling the crude oil to separate naphtha, and mixing the naphtha and 2,3-dimethyl-2-butene.

This application is based on and claims the benefit of priority from Japanese Patent Application No. 2022-126383, filed on 8 Aug 2022, the content of which is incorporated herein by reference.

### BACKGROUND OF THE INVENTION

### Field of the Invention

The present invention relates to a method for producing a gasoline alternative fuel and a gasoline alternative fuel.

### Related Art

Effort is being made for the purpose of mitigating climate change or reducing the influence thereof, and research and development on reduction of carbon dioxide emission are being carried out. Among them, e-fuel is drawing attention. E-fuel is a synthetic fuel synthesized using hydrogen generated by electrolysis of water using electrical energy derived from renewable energy such as sunlight, wind power, etc.

As an example of a method for producing a synthetic fuel, there is known a method in which carbon monoxide is reacted with hydrogen to synthesize a crude oil by a Fischer-Tropsch (FT) method, and then the crude oil is distilled. Thus, diesel fuel and jet fuel can be obtained, but it is desirable to effectively use naphtha, which is a by-product.

As another example of a method for producing a synthetic fuel, there is known a method including synthesizing methanol by reacting carbon monoxide and/or carbon dioxide with hydrogen, and then synthesizing a gasoline alternative fuel from methanol by an MTG method (for example, see Patent Document 1).

Patent Document 1: Japanese Unexamined Patent Application, Publication No. 2015-44926

### SUMMARY OF THE INVENTION

However, it is desirable to reduce the amount of carbon dioxide emission when producing a gasoline alternative fuel. Therefore, it is conceivable to use naphtha produced by the FT method when producing the gasoline alternative fuel, but it is necessary to set research octane number of the gasoline alternative fuel to 89 or more.

It is an object of the present invention to provide a method for producing a gasoline alternative fuel capable of reducing an amount of carbon dioxide emission, and a gasoline alternative fuel.

(1) A method for producing a gasoline alternative fuel, including: synthesizing a crude oil by a Fischer-Tropsch method, distilling the crude oil to separate naphtha, and mixing the naphtha and 2,3-dimethyl-2-butene.

(2) The method for producing a gasoline alternative fuel as described in aspect (1), further including synthesizing propylene from methanol by an MTP method; and synthesizing 2,3-dimethyl-2-butene by dimerizing the propylene.

(3) The method for producing a gasoline alternative fuel as described in aspect (2), in which instead of synthesizing propylene from methanol by the MTP method, the propylene is obtained by cracking naphtha.

(4) A method for producing a gasoline alternative fuel, including synthesizing a crude oil by a Fischer-Tropsch method, distilling the crude oil to separate light naphtha and heavy naphtha, cracking the heavy naphtha to obtain propylene, dimerizing the propylene to synthesize 2,3-dimethyl-2-butene, and mixing the light naphtha and the 2,3-dimethyl-2-butene.

(5) A gasoline alternative fuel including naphtha produced by a Fischer-Tropsch method and 2,3-dimethyl-2-butene.

(6) The gasoline alternative fuel as described in aspect (5), in which the naphtha is light naphtha.

According to the present invention, it is possible to provide a method for producing a gasoline alternative fuel capable of reducing an amount of carbon dioxide emission, and a gasoline alternative fuel.

### BRIEF DESCRIPTION OF THE DRAWINGS

FIG. 1 is a diagram showing an example of the first producing method of a gasoline alternative fuel of the present embodiment;

FIG. 2 is a diagram showing another example of the first producing method of the gasoline alternative fuel of the present embodiment; and

FIG. 3 is a diagram showing the second producing method of the gasoline alternative fuel of the present embodiment.

### DETAILED DESCRIPTION OF THE INVENTION

Hereinafter, embodiments of the present invention will be described with reference to the accompanying drawings.

### [Method For Producing Gasoline Alternative Fuel]

### (First Method)

The first producing method of the gasoline alternative fuel of the present embodiment includes: synthesizing a crude oil by a Fischer-Tropsch (FT) method, distilling the crude oil to separate naphtha, and mixing naphtha and 2,3-dimethyl-2-butene. Here, since the crude oil can be synthesized using hydrogen generated by electrolysis of water using electric energy derived from renewable energy, the amount of carbon dioxide emission can be reduced. Since naphtha and 2,3-dimethyl-2-butene are mixed, research octane number of the gasoline alternative fuel can be set to 89 or more. Here, the research octane number of naphtha is, for example, 50 or more and less than 80.

Table 1 shows the research octane number (RON; JIS K2280-1) of a mixture of a standard fuel (75 volume %) and olefin (25 volume %). Here, as the standard fuel, a mixture (PRF65) of n-heptane (35 volume %) and isooctane (65 volume %) was used.

TABLE 1

| | 2-methyl-1-butene | 2-methyl-2-butene | 3,3-dimethyl-1-butene | 2,3-dimethyl-1-butene | 2,3-dimethyl-2-butene |
|---|---|---|---|---|---|
| Chemical formula | CH₂CH₂C(CH₃)═CH₂ | [symbol] CH═C [symbol] | [symbol] CCH═CH₂ | [symbol] ═C( [symbol] )CH [symbol] | (CH₃)₂═C(CH₃)₂ |
| RON | 84.9 | 86.4 | 80.0 | 82.4 | 89.7 |

[symbol]

indicates data missing or illegible when filed

From Table 1, it is understood that the effect of improving RON tends to increase as a proportion of C—C═C— structure included in the olefin increases, and 2,3-dimethyl-2-butene having four C—C═C═ structures has the largest effect of improving RON.

The volume ratio of 2,3-dimethyl-2-butene with respect to naphtha is not particularly limited as long as the research octane number of the gasoline alternative fuel can be set to 89 or more, but is, for example, ⅓ or more and 1 or less.

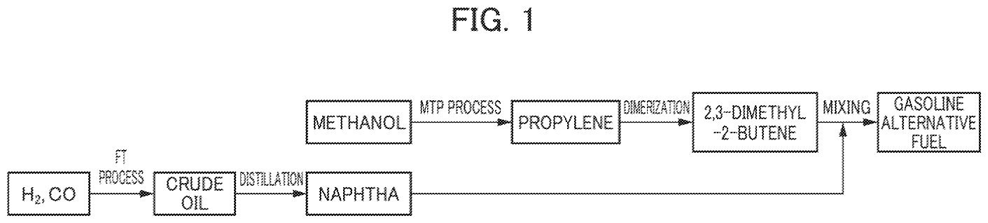

The first production method of the gasoline alternative fuel of the present embodiment preferably further includes synthesizing propylene from methanol by an MTP method, and synthesizing 2,3-dimethyl-2-butene by dimerizing propylene (see FIG. 1). Here, since methanol can be synthesized using hydrogen generated by electrolysis of water using electric energy derived from renewable energy, the amount of carbon dioxide emission can be reduced.

As a method for synthesizing 2,3-dimethyl-2-butene by dimerizing propylene, a well-known method can be used (See, e.g., Yuhki Gohsei Kagaku (Organic Synthesis Chemistry), Vol. 48, No. 9 (1990), pp. 806-813.).

When 2,3-dimethyl-2-butene is synthesized, by-products such as 2,3-dimethyl-1-butene, 4-methyl-2-pentene, 2-methyl-1-pentene, and 2-methyl-2-pentene are produced in some cases. In these cases, the gasoline alternative fuel may contain the above-mentioned by-products as long as the research octane number is 89 or more. That is, in the first production method of the gasoline alternative fuel of the present embodiment, since the fractionation step of removing the by-products from 2,3-dimethyl-2-butene can be omitted, the amount of carbon dioxide emission can be reduced.

Note that propylene derived from petroleum may be used by omitting synthesizing propylene from methanol by the MTP method.

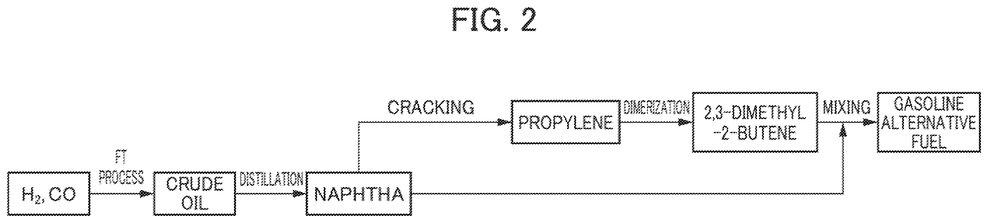

Alternatively, instead of synthesizing propylene from methanol by the MTP method, a step of synthesizing crude oil by an FT method, a step of distilling the crude oil to separate naphtha, and a step of cracking naphtha to obtain propylene may be performed (see FIG. 2). Here, since the crude oil can be synthesized using hydrogen generated by electrolysis of water using electric energy derived from renewable energy, the amount of carbon dioxide emission can be reduced.

Naphtha can be cracked in the same manner as the cracking of petroleum naphtha.

### (Second Method)

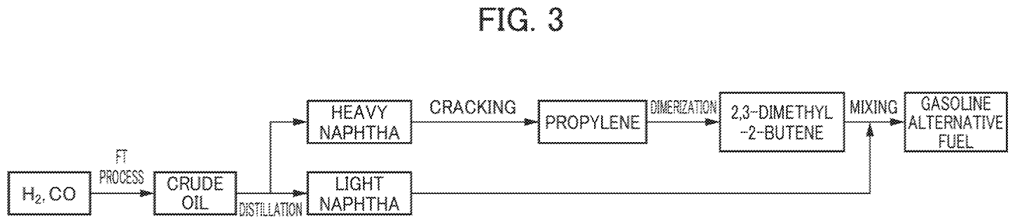

The second production method of the gasoline alternative fuel of the present embodiment includes a step of synthesizing a crude oil by a Fischer-Tropsch method, a step of separating light naphtha and heavy naphtha by distillation of the crude oil, a step of cracking the heavy naphtha to obtain propylene, a step of synthesizing 2,3-dimethyl-2-butene by dimerizing propylene, and a step of mixing the light naphtha and 2,3-dimethyl-2-butene (see FIG. 3). Here, since the crude oil can be synthesized using hydrogen generated by electrolysis of water using electric energy derived from renewable energy, the amount of carbon dioxide emission can be reduced. Further, since the light naphtha and 2,3-dimethyl-2-butene are mixed, the research octane number of the gasoline alternative fuel can be set to 89 or more. Here, the research octane number of light naphtha is, for example, 70 or more and less than 80, and the research octane number of heavy naphtha is, for example, 60 or more and less than 70. Therefore, the volume ratio of 2,3-dimethyl-2-butene with respect to light naphtha can be reduced. Further, since the heavy naphtha is cracked to obtain propylene, the heavy naphtha can be effectively utilized.

After the crude oil is distilled to separate naphtha, the naphtha may be distilled to separate the light naphtha and the heavy naphtha.

### [Gasoline Alternative Fuel]

The gasoline alternative fuel of the present embodiment includes naphtha produced by the FT method and 2,3-dimethyl-2-butene. Here, naphtha produced by the FT method can be separated from crude oil synthesized by the FT method. Further, since the crude oil synthesized by the FT method can be synthesized using hydrogen generated by electrolysis of water using electric energy derived from renewable energy, the amount of carbon dioxide emission can be reduced.

The content of 2,3-dimethyl-2-butene in the gasoline alternative fuel of the present embodiment is not particularly limited as long as the research octane number can be set to 89 or more, but is, for example, 25 volume % or more and 50 volume % or less.

The gasoline alternative fuel of the present embodiment can be produced by the first production method of the gasoline alternative fuel of the present embodiment.

The naphtha produced by the FT method is preferably light naphtha. Thereby, the content of 2,3-dimethyl-2-butene in the gasoline alternative fuel of the present embodiment can be reduced. In this case, the gasoline alternative fuel of the present embodiment can be produced by the second manufacturing method of the gasoline alternative fuel of the present embodiment.

Although the embodiments of the present invention have been described above, the present invention is not limited to the above-described embodiments, and the above-described embodiments may be appropriately modified within the scope of the present invention.

What is claimed is:

1. A method for producing a gasoline alternative fuel, comprising:
synthesizing a crude oil by a Fischer-Tropsch method,
distilling the crude oil to separate naphtha, and
mixing the naphtha and 2,3-dimethyl-2-butene.

2. The method for producing a gasoline alternative fuel according to claim 1,
the method further comprising synthesizing propylene from methanol by an MTP method,
and synthesizing 2,3-dimethyl-2-butene by dimerizing the propylene.

3. The method for producing a gasoline alternative fuel according to claim 2, wherein instead of synthesizing propylene from methanol by the MTP method, propylene is obtained by cracking naphtha.

4. A method for producing a gasoline alternative fuel, comprising:
synthesizing a crude oil by a Fischer-Tropsch method,
distilling the crude oil to separate light naphtha and heavy naphtha,
cracking the heavy naphtha to obtain propylene,
dimerizing the propylene to synthesize 2,3-dimethyl-2-butene, and
mixing the light naphtha and the 2,3-dimethyl-2-butene.

5. A gasoline alternative fuel comprising naphtha produced by a Fischer-Tropsch method and 2,3-dimethyl-2-butene.

6. The gasoline alternative fuel according to claim 5, wherein the naphtha is light naphtha.
